(19) United States
(12) Patent Application Publication
(10) Pub. No.: US 2004/0157254 A1
(43) Pub. Date: Aug. 12, 2004

(54) System and method for designing probes using heterogeneous genetic information, and computer readable medium

(76) Inventor: Taejoon Kwon, Gyeonggi-do (KR)

Correspondence Address: Cantor Colburn LLP, 55 Griffin South Road, Bloomfield, CT, 06002, US

(21) Appl. No.: 10/773,507

(22) Filed: Feb. 5, 2004

(30) Foreign Application Priority Data
Feb. 5, 2003 (KR) 2003-7122

(51) Int. Cl. C12Q 1/68; G06G 7/48; G06G 7/58; G06F 19/00; G01N 33/48; G01N 33/50

(52) U.S. Cl. 435/6; 702/20; 703/11

(57) ABSTRACT

Provided are a probes design system and method using heterogeneous genetic information. A storage unit stores a crosslink map having records according to the version of a genome sequence present the outside of the design system. An information search unit searches for the identifier and sequence information corresponding to target genetic information among the genetic information about the genome sequence in the crosslink map. A location estimation unit determines a reference group made up of reference genetic information which is contained in more than a predetermined number in an organism, calculates difference values of the start positions and the end positions of the reference genetic information based on the crosslink map, and determines the location of the target genetic information on the latest genome sequence by a location shift corresponding to the difference values.

### BACKGROUND OF THE INVENTION

This application claims the priority of Korean Patent Application No. 2003-7122, filed on Feb. 5, 2003, in the Korean Intellectual Property Office, the disclosure of which is incorporated herein by reference in its entirety.

1. Field of the Invention

The present invention relates to a system and method for designing a probes, and more particularly, to a system and method for designing an oligonucleotide probes using heterogeneous data sources.

2. Description of the Related Art

There has been an increasing interest on an oligonucleotide microarray offering abundant biological information through a fewer number of experiments. Acquisition of biological information using the oligonucleotide microarray is possible by determining the binding affinity of a target gene to support material-bound oligonucleotides. In this case, the support material-bound oligonucleotides serve as probes. The target sample, whose biological information wants to be found, is obtained by PCR with labeling.

Recently, the microarray technology has been widely applied in expression profile analysis for determining gene expression profile and genotyping analysis for determining specific genomic gene information. A large number of oligonucleotide probes with a shorter length than a sample gene are used both in the expression profile analysis and the genotyping analysis.

Meanwhile, selection of optimal oligonuleotide probe set is an important process in microarray experiments. Generally, desired probes are selected by predicting the binding affinity between a sample gene and candidate probes expected to bind with the sample gene. Since exact prediction enables to construction of a high performance microarray with small efforts, there have been done many studies on developing more exact prediction systems for the binding affinity between a sample gene and probes, and optimal probe selection methods based on such prediction systems.

However, management of information related to selected probes is not an easy question. In particular, the preliminary annotation of human genome is already available but is being continually refined. In this regard, genetic information (for example, variation information such as polymorphism and gene function-related information) associated with the human genome sequence will be continually modified. Under these circumstances, previously designed probes may now be related to genetic information different from the genetic information at the time of probe design. For example, this is true in a case where a new polymorphism is found or there is a binding potential between probes and newly known genome sequences. In this case, if no relationship between probe information at the time of probe design and the latest probe information is given, it is difficult to acquire the latest probe information from the previous probe information.

Many studies on the oligonucleotide probe selection method have been done because it can be applied in the hybridization technology or the PCR primer design technology, in addition to the microarray technology.

Australia Patent No. AU7,534,901 discloses a technology for predicting hybridization thermodynamics using a database related to thermodynamic parameters for sequence information, hybridization level information, and correction information. That is, this patent relates to a method for accurately predicting nucleotide hybridization, and thus, can be applied in construction of an accurate probe design system. However, this patent fails to disclose data search based on designed probes and efficient management of relational information.

Meanwhile, European Patent No. EP1,103,910 discloses a method for automatically selecting oligonucleotide probes that hybridize with a template sequence containing a region intended for genotyping. In particular, this patent relates to a probe design method for detecting a mutation on DNA. The probe design method includes defining the mutation site between a wild-type sequence and a mutant-type sequence, and selecting oligonucleotide probes which have a one-to-one hybridization fit to the two sequences extended from the mutation site in both directions. This method can be applied in selection of optimal oligonucleotide probes for detecting a mutation site. However, this patent is silent about management of designed probes, a mutant-type sequence, and a wide-type sequence.

U.S. Pat. No. 6,403,314 discloses a computational method and system for predicting fragmented hybridization and for identifying potential cross-hybridization. This patent relates to a method for predicting cross-hybridization potential of non-target samples and probes by considering all possible pairing in a probe unit and a sample unit. Cross-hybridization of non-target samples and probes is one of main factors determining probe's ability and must be considered upon a probe design. However, unless information between a sample unit and a probe unit and between a sample unit and another sample unit is efficiently managed, even when the sample unit or the probe unit is slightly modified, previously compiled information becomes useless.

U.S. Pat. No. 6,251,588 discloses a method for estimating an oligonucleotide probe sequence. That is, this patent relates to a method for predicting the potential of hybridization using a clustering technology and determining the ranking of candidate probes. While the above-described patents generally focus on a method for accurately predicting the thermodynamic properties of probes, this patent has been made from a broader point of view. That is, this patent focuses on selecting efficient candidate probe sets based on estimated probe properties. However, this patent is also silent about efficient information management, like the above-described patents.

In summary, probe design-related prior arts are interest in accurate prediction of probe characteristics and selection of good probes based on the accurate prediction. However, under such circumstances that relational information continues to be edited and new information continues to be disclosed, it is important to efficiently manage designed probe information for easy search of relational information, as well as to accurately design probes.

U.S. Pat. No. 6,188,783 discloses a method and system for providing a probes chip database. This patent relates to a database management of relationship between probes and samples. However, when one probe information is associated with several samples, no mention is made about new relationship between the probe and the samples based on interrelationship of the samples. That is, even though probe and sample information is managed in a relational database, failure to define sample-to-sample relationship complicates the finding of the interrelationship between previous probe information and the latest sample information.

Meanwhile, since bioinformatics deal with various data types, data integration is very important. There are many patent documents regarding such data integration. The present invention is associated with effective exploitation of desired information through efficient management of probe and sample sequence information. In this regard, there is need to review data integration-related technologies.

WO01/55911 discloses an integrated access system to biomedical resources. That is, this patent relates to a data processing system for allowing for communication between a local database system and a remote database system using a data object linker, a data object determinant, and a graphical user interface (GUI) enabling a user to view data objects graphically.

WO02/39486 discloses an integrated system for bioinformatics information. That is, this patent relates to a method for integrating an interface based object data model using a client bus that is responsible for communication between client environment with user interface (Ul) and software components.

WO01/01294 discloses a biological data processing. According to this patent, several databases or servers receive queries in a structured format such as XML, and a translation server translates the received queries into commands recognized by each server.

European Patent No. EP1,215,614 discloses a method for recording a gene analysis data. According to this patent, gene variation information found by an experimental analysis method such as sequencing is recorded on a recording medium together with reference information. Even though data items and recording method are listed, there is no mention about a method for storing and managing data in a specific format.

The general purpose of the above-described patents is to provide data integration technology. That is, the above-described patents provide efficient linkage of relational data when a specific task such as searching is carried out. However, there is no mention about searching for previous information and interlinking between the previous information and the latest information.

### SUMMARY OF THE INVENTION

The present invention provides a system and method for designing a novel oligonucleotide probes based on probe information that has already been designed, through integration of information necessary for oligonucleotide probe design, in the field of studies and diagnoses that exploit microarray experimental results.

The present invention also provides a computer readable medium having embodied thereon a computer program for a method for designing a novel oligonucleotide probes based on probe information that has already been designed, through integration of information necessary for oligonucleotide probe design, in the field of studies and diagnoses that exploit microarray experimental results.

According to an aspect of the present invention, there is provided a system for designing a probes using heterogeneous genetic information, comprising: a storage unit storing a crosslink map having records according to the version of a genome sequence; an information search unit searching for the identifier and sequence information corresponding to target genetic information among the genetic information about the genome sequence in the crosslink map; and a location estimation unit determining a reference group made up of reference genetic information which is contained in more than a predetermined number in an organism, calculating difference values of the start positions and the end positions of the reference genetic information based on the crosslink map, and determining the location of the target genetic information on the latest genome sequence by a location shift corresponding to the difference values.

According to another aspect of the present invention, there is provided a method of designing a probes using heterogeneous genetic information, comprising: creating a crosslink map having records according to the version of a genome sequence; searching for the identifier and sequence information corresponding to target genetic information among the genetic information about the genome sequence in the crosslink map; determining a reference group made up of reference genetic information which is contained in more than a predetermined number in an organism; calculating difference values of the start positions and the end positions of the reference genetic information based on the crosslink map; and determining the location of the target genetic information on the latest genome sequence by a location shift corresponding to the difference values.

Therefore, the latest information about probes that have recently been designed can be obtained. Even though the genome sequence information and the identifier information at the time of probe design are not the latest information at present time, the latest information can be searched for from a crosslink map.

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram that illustrates a probes design system according to an embodiment of the present invention;

FIG. 2 is a view that illustrates an example of a crosslink map;

FIG. 3 is a block diagram that illustrates the detailed construction of a location estimation unit;

FIG. 4 is a flowchart that illustrates a probe design method according to the present invention;

FIG. 5 is a flowchart that illustrates a location estimation process in a probe design method according to the present invention; and

FIG. 6 is a view that illustrates the start and end positions of identifier information in the previous sequence and the latest sequence of BRCA2 target gene information acquired from a crosslink map.

### DETAILED DESCRIPTION OF THE INVENTION

Hereinafter, a system and method for designing a probes according to the present invention will be described in detail with reference to the accompanying drawings.

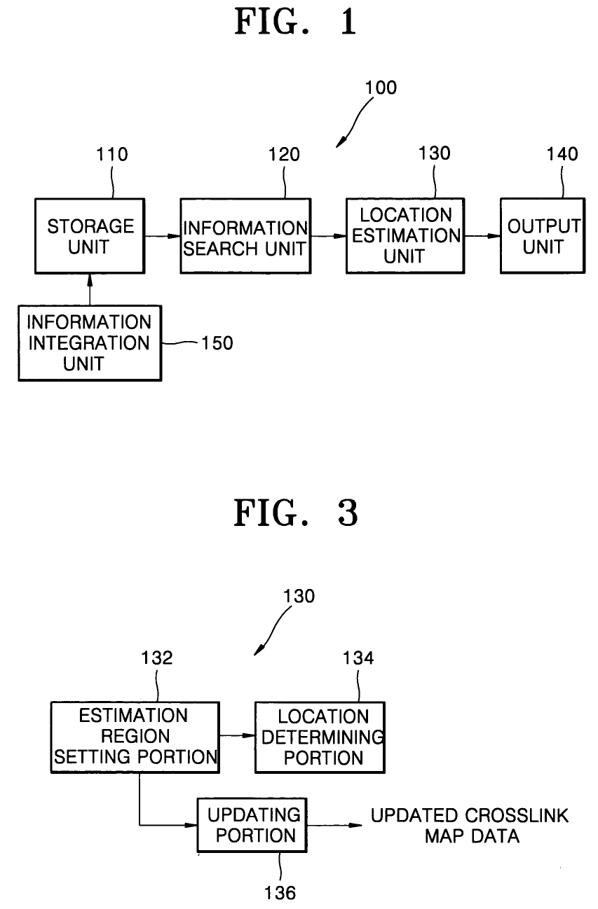

FIG. 1 is a block diagram that illustrates a probes design system according to the present invention.

Referring to FIG. 1, a probes design system 100 according to the present invention includes a storage unit 110, an information search unit 120, a location estimation unit 130, an output unit 140, and an information integration unit 150.

The storage unit 110 stores a crosslink map defining the interrelationship among different sources. The crosslink map is created in the form of a table that defines the interrelationship among different sources using various identifier information. For example, with respect to genome version 1.0 and genome version 2.0, the crosslink map stores cross-referable information based on the location information of identifiers between the two genome versions, such as protein, coding sequence (CDS), exon/intron, regulatory region, mRNA, sequence tagged site (STS), expressed sequence tag (EST), and contig sequence.

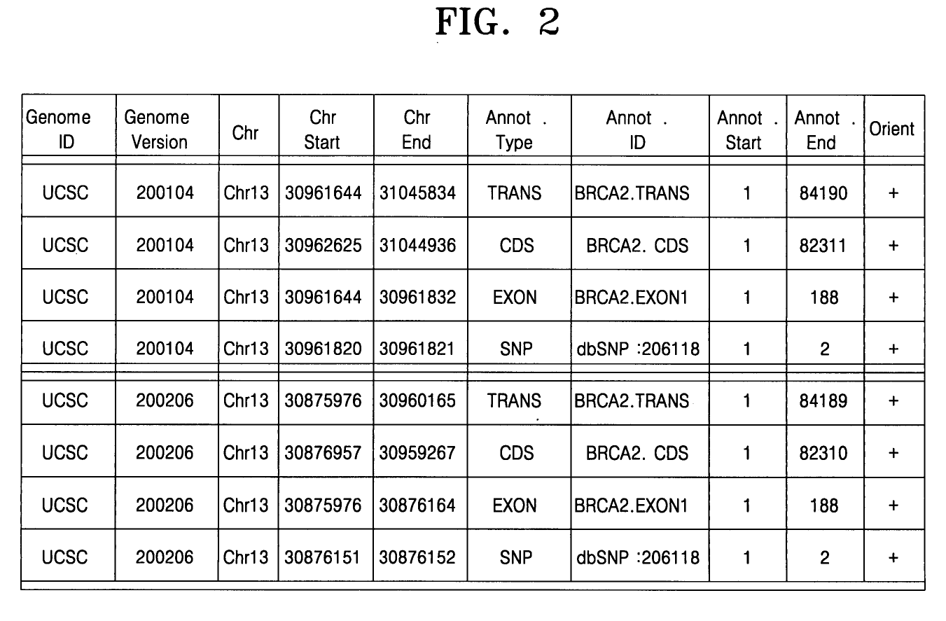

Here, the location information can be considered according to individual identifier information. For example, the location information is information related to the relative locations of genome sequences on different sources on the basis of chromosome information for chromosome-specific probes, contig information, related mRNA, surrounding known STS, EST, and exon/intron. Considering location information based on various identifier information is the most important task for information comparison among different genome sequences. Here, the different genome sequences may be transformed into data formats in the same manner or in a completely different manner on the basis of new information. The difference between build 28 information and build 29 information from the National Center for Biotechnology information (NCBI) corresponds to the former, and the difference between the NCBI information and the University Classified Staff Council (UCSC) information corresponds to the latter. FIG. 2 shows an example of a crosslink map.

The information search unit 120 searches for information based on the interrelationship of identifier information displayed in a crosslink map. Once a request for specific information is received, the information search unit 120 functions to search for and output all relational information. For example, when a request for the mRNA of BRCA2 gene is received, the information search unit 120 searches for and extracts all records on the crosslink map containing corresponding information. If the requested mRNA information is about a specific genome assembly version, the information search unit 120 searches for information on the crosslink map and determines whether the searched information is the latest one. If the searched information is not the latest one, a message indicating that information is updated is output as well.

When various results for requested single information are released, the location estimation unit 130 predicts an exact result by assigning a priority order to the results. Location information for specific genetic information in the crosslink map is recorded according to different standards. Therefore, the location estimation unit 130 can exactly predict a result by assigning a priority order to various results for single information. That is, as exemplified above, when a request for the mRNA of BRCA2 gene is received, the location estimation unit 130 searches for the location of corresponding information in the crosslink map according to different standards such as chromosome, related contig, coding sequence, protein sequence, and exon/intron information.

In this case, the location estimation unit 130 selects genetic information which is contained in more than a predetermined number in an organism as reference genetic information and determines a reference group made up of such genetic information. Then, the location estimation unit 130 calculates difference values of the start positions and the end positions of the reference genetic information based on the crosslink map, and then determines the location of target genetic information by a location shift corresponding to the difference values. Generally, exons and protein-encoding sequences are contained in all genomes of a specific organism. A location change of these genetic information little occurs even when genome sequence version is updated. The location estimation unit 130 determines, as reference genetic information, genetic information whose location is little changed even when genome version is updated. A priority is given to the difference values of the start positions and the end positions of genetic information which is contained in a more number in an organism among the reference genetic information. The location of the target genetic information is determined based on the difference values.

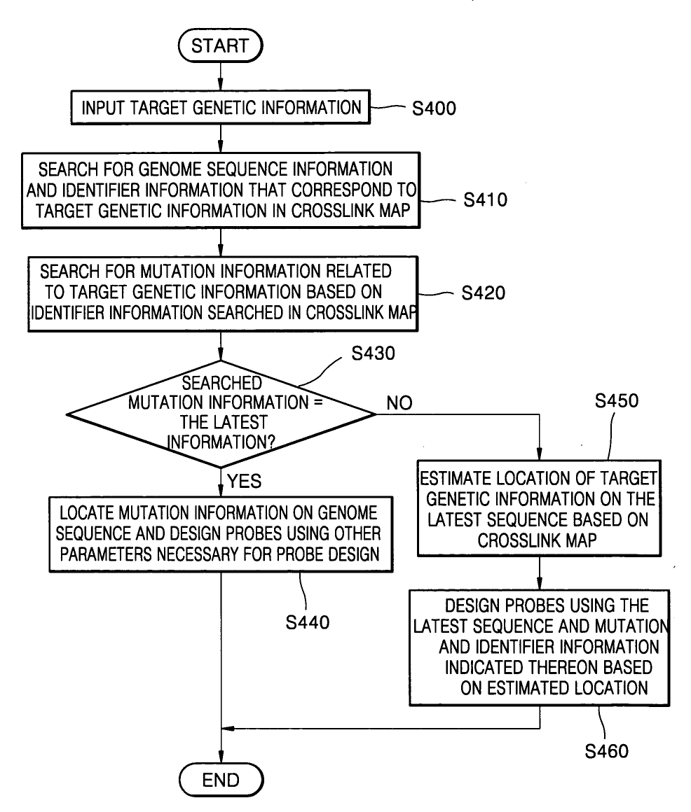

Meanwhile, the location estimation unit 130 may set a region at which the target genetic information may be present and then determine the location of the target genetic information in the region. In this case, the location estimation unit 130 includes an estimation region setting portion 132 and a location determining portion 134. The detailed structure of the location estimation unit 130 is shown in FIG. 3. The estimation region setting portion 132 calculates difference values of the start positions and the end positions of genetic information excluded from the reference group based on the crosslink map. Then, the estimation region setting portion 132 sets an estimation region for the location of the target genetic information on the latest genome sequence based on the difference values thus calculated. The location determination portion 134 determines the location of the target genetic information in the estimation region by a location shift corresponding to the difference values calculated with respect to the reference genetic information.

The location estimation unit 130 also includes an updating portion 136 that updates the reference group based on the crosslink map. The updating portion 136 updates the reference group as follows: calculating difference values of the start positions and the end positions of genetic information which is commonly present on individual genome versions and then selecting genetic information in which the calculated difference values are within a predetermined range. The updating portion 136 calculates the difference values of the start positions and the end positions of genetic information which is commonly present on individual genome versions. Alternatively, the difference values may be calculated in the estimation region setting portion 132 and then input into the updating portion 136.

The output unit 140 outputs the difference between the requested information and the latest information. When a request for different genome versions is received, location information for the same genetic information on the crosslink map may not be identical. For example, in comparison with the shift of 10,000 bp (base pairs) on a chromosome, 9,900 bp may be shifted on a contig. Generally, the location information of function-related protein sequence and exon sequence is well preserved as compared to that of chromosome or contig information. In this regard, based on genetic information whose position information is better preserved, an exact location can be searched even between different genome versions.

The information integration unit 150 receives information necessary for the crosslink map from various information sources. For this, there is required an element executing the transformation of individual data formats into data formats recognized by the crosslink map. The information integration unit 150 transforms previous data into new data formats whenever new data is considered. Even when data is present in various databases, the information integration unit 130 accesses to a corresponding data server, receives corresponding data, and transforms the corresponding data into a desired data format.

FIG. 4 is a flowchart that illustrates a probe design method according to the present invention.

Referring to FIG. 4, first, target genetic information for probe design is input (step S400). At this time, in a case where an object of the probe design is to detect sequence mutation, in addition to the target genetic information, related mutation information is also input.

The information search unit 120 searches for genome sequence information and identifier information that correspond to the target genetic information in a crosslink map (step S410). Then, the information search unit 120 searches for mutation information related to the target genetic information based on the identifier information searched in the crosslink map (step S420). Then, the information search unit 120 checks whether the searched mutation information and identifier information are the latest one (step S430). Since the crosslink map always contains the latest information, whether the searched information is the latest one can be easily determined even when only information recorded in the crosslink map is used.

When the searched mutation and identifier information are the latest one, according to a common probe design method, the mutation information is located on the genome sequence and various other parameters necessary for the probe design are received (step S440). However, when the searched mutation and identifier information is not the latest one, a probe design cannot be commenced at once. In this case, the location estimation unit 130 estimates the location of the target genetic information on the latest sequence based on corresponding mutation and identifier information in the crosslink map (step S450). Subsequent to such estimation, probes are designed using the latest sequence and the mutation and identifier information indicated thereon (step S460).

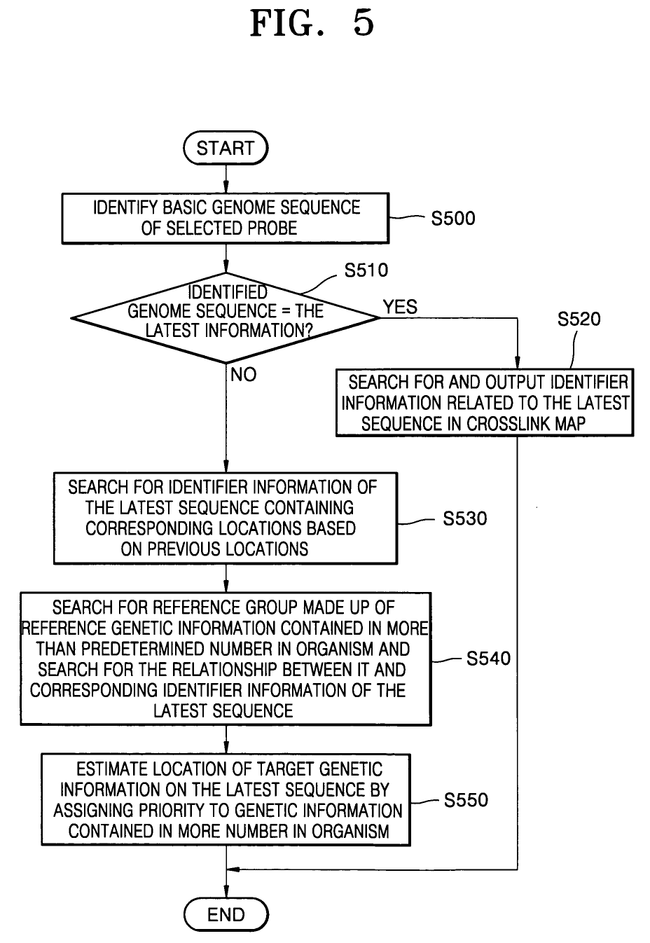

FIG. 5 is a flowchart that illustrates a location estimation process in a probe design method according to the present invention.

Referring to FIG. 5, the information search unit 120 selects probes whose information is to be searched and identifies a genome sequence from which the probes have been designed (step S500). The identified information is managed as probe-related information. Also, the information search unit 120 determines whether the genome sequence is based on the latest information (step S510).

If the probes are those designed from the latest sequence, the information search unit 120 searches for and outputs identifier information related to the latest sequence in the crosslink map (step S520). In this case, other type information such as previous sequence or protein sequence based on crosslink map information is also output, together with the latest sequence information.

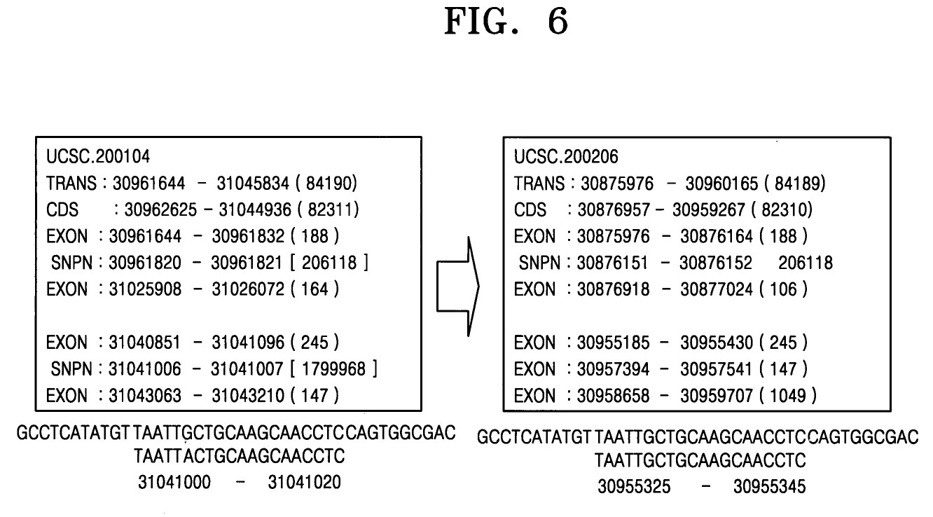

On the other hand, if the probes are those designed from the previous sequence, the location estimation unit 130 estimates the locations of the probes on the latest sequence based on the crosslink map. First, the location estimation unit 130 receives the identifier information of the latest sequence containing corresponding locations based on previous locations searched in the information search unit 120 (step S530). Examples of the identifier information searched in the information search unit 120 include contig, exon, protein sequence, and mRNA of the previous sequence on which the probes have been located. FIG. 6 shows the start positions and the end positions of the identifier information in the previous sequence and the latest sequence of target genetic information, BRCA2, based on the crosslink map.

Next, the location estimation unit 130 searches for the relationship between the received identifier information and corresponding identifier information of the latest sequence (step S540). For this, the location estimation unit 130 calculates difference values of the start positions and the end positions of corresponding identifier information in the previous sequence and the latest sequence. Referring to FIG. 6, it can be seen that difference values of remaining genetic information except for SNPN are within the range of −85668 to −85669.

In this case, various identifier information may be contradicted. For example, a position difference between contigs and a position difference between exons may be different. At this time, the location estimation unit 130 synthesizes information by assigning a priority to more reliable information and estimates the location of the target genetic information on the latest sequence using location change information predicted as the most exact information (step S550). In this case, generally, the location of the target genetic information is estimated based on exons that are contained in all genome sequences of an organism. In this regard, since exons present on UCSC.200104 version are shifted within a range of −85668 to −85669, it is estimated that SNPN present on UCSC.200104 is not present on UCSC.200206 version. If differences between identifier information are too large to predict a location, a message indicating such fact is output. A message indicating that mapping is impossible may also be output.

In step S550, the location estimation unit 130 may set a potential estimation region based on difference values of the start positions and the end positions of the corresponding identifier information in the previous sequence and the latest sequence. In this case, second exon of FIG. 6 is genetic information having the largest location change, as −148990 to −149048. Therefore, the location estimation unit 130 estimates the location of the target genetic information within a range of the difference values of the second exon based on difference values calculated with respect to reference genetic information.

When probes are mapped on the latest sequence according to the above-described manner, the latest identifier information related to corresponding regions can be obtained. Also, when these information are synthetically utilized, various types of the latest information about corresponding probes can be obtained. Therefore, even though users for probe information do not design new probes, information corresponding to that obtainable from newly designed probes can be obtained. For example, in comparison between micro array information constructed 3 years ago and the latest micro array information, individual probes may be changed or related information of the same probes may be changed. Conventionally, the locations of probes are separately searched in the latest information. Of course, the information of similar regions can be searched using related information such as gene name. However, additional efforts are required to search for exact location information. The probe design system according to the present invention can exactly predict location information using various identifier information defined in the previous information and the latest information without additional efforts.

In addition, in a case where there are various micro array experimental results for one subject (for example, specific gene), by using a function of searching for probe information of the present invention, previous experimental information and the latest experimental information can be compared and the latest related information for the previous experimental information can be searched. For example, in the case of designing probes for disease diagnosis, related information about a genome sequence can be obtained by comparing related information acquired from separate database managing disease-related mutation and genetic information based on the crosslink map. Therefore, information related to probe design can be easily managed. Also, even when specific information is named based on the previous information, it can be compared with the latest information.

The present invention can be embodied as a computer readable code on a computer readable medium. The computer readable medium includes all types of recording medium storing data readable by computer system. For example, the computer readable medium includes ROMs, RAMs, CD-ROMs, magnetic tapes, floppy disks, optical data storage media, and carrier waves (e.g., transmissions over the Internet). Also, the computer readable medium may store computer readable codes distributed in computer systems connected by a network so that a computer can read and execute the codes in a distributed manner.

According to a probe design system and method of the present invention, the latest information about probes that have recently been designed can be obtained. Even though the genome sequence information and the identifier information at the time of probe design are not the latest information at present time, the latest information can be searched for from a crosslink map. Also, the present invention can be utilized in a case where probe information is continuously changed to enhance micro array properties. Furthermore, since genome sequence information or related identifier information is isolated from probe design means, fully managed external data can be utilized.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A system for designing a probes using heterogeneous genetic information, comprising:
a storage unit storing a crosslink map having records according to the version of a genome sequence;
an information search unit searching for the identifier and sequence information corresponding to target genetic information among the genetic information about the genome sequence in the crosslink map; and
a location estimation unit determining a reference group made up of reference genetic information which is contained in more than a predetermined number in an organism, calculating difference values of the start positions and the end positions of the reference genetic information based on the crosslink map, and determining the location of the target genetic information on the latest genome sequence by a location shift corresponding to the difference values.

2. The system of claim 1, further comprising an information integration unit receiving data corresponding to entries recorded on the crosslink map from various sources about the genome sequence and transforming the received data into data formats recognized by the crosslink map.

3. The system of claim 1, wherein entries recorded on the crosslink map comprises a name of the genome sequence, a version of the genome sequence, an identifier of the genetic information about the genome sequence, a start position and an end position of the genetic information on the genome sequence, and a length of the genetic information about the genome sequence.

4. The system of claim 1, wherein the location estimation unit determines the location of the target genetic information by assigning a priority to the difference values calculated with respect to genetic information which is contained in a more number in an organism.

5. The system of claim 1, wherein the location estimation unit comprises:
an estimation region setting portion calculating difference values of the start positions and the end positions of genetic information excluded from the reference group based on the crosslink map and setting an estimation region for the location of the target genetic information on the latest genome sequence based on the calculated difference values; and
a location determining portion determining the location of the target genetic information in the estimation region of the latest genome sequence by a location shift corresponding to the difference values calculated with respect to the reference genetic information.

6. The system of claim 1, wherein the location estimation unit further comprises an updating portion updating the reference group in such a way to calculate difference values of the start positions and the end positions of genetic information which is commonly present on individual versions of the genome sequence and select genetic information in which the calculated difference values are within a predetermined range.

7. A method of designing a probes using heterogeneous genetic information, the method comprising:
creating a crosslink map having records according to the version of a genome sequence;
searching for the identifier and sequence information corresponding to target genetic information among the genetic information about the genome sequence in the crosslink map;
determining a reference group made up of reference genetic information which is contained in more than a predetermined number in an organism;
calculating difference values of the start positions and the end positions of the reference genetic information based on the crosslink map; and
determining the location of the target genetic information on the latest genome sequence by a location shift corresponding to the difference values.

8. The method of claim 7, wherein entries recorded on the crosslink map comprises a name of the genome sequence, a version of the genome sequence, an identifier of the genetic information about the genome sequence, a start position and an end position of the genetic information on the genome sequence, and a length of the genetic information about the genome sequence.

9. The method of claim 7, wherein determining the location of the target genetic information is carried out by assigning a priority to the difference values calculated with respect to genetic information which is contained in a more number in an organism.

10. The method of claim 7, further comprising updating the reference group in such a way to calculate difference values of the start positions and the end positions of genetic information which is commonly present on individual versions of the genome sequence and select genetic information in which the calculated difference values are within a predetermined range.

11. The method of claim 7, wherein determining the location of the target genetic information comprises:
calculating difference values of the start positions and the end positions of genetic information excluded from the reference group based on the crosslink map and setting an estimation region for the location of the target genetic information on the latest genome sequence based on the calculated difference values; and
determining the location of the target genetic information in the estimation region of the latest genome sequence by a location shift corresponding to the difference values calculated with respect to the reference genetic information.

12. A computer readable medium having embodied thereon a computer program for a method of designing a probes using heterogeneous genetic information, the method comprising:
creating a crosslink map having records according to the version of a genome sequence;
searching for the identifier and sequence information corresponding to target genetic information among the genetic information about the genome sequence in the crosslink map;
determining a reference group made up of reference genetic information which is contained in more than a predetermined number in an organism;
calculating difference values of the start positions and the end positions of the reference genetic information based on the crosslink map; and
determining the location of the target genetic information on the latest genome sequence by a location shift corresponding to the difference values.
